(19) United States
(12) Patent Application Publication
(10) Pub. No.: US 2025/0345489 A1
(43) Pub. Date: Nov. 13, 2025

(54) POROUS COMPOSITE MATERIAL, MANUFACTURING METHOD THEREOF AND BONE SUBSTITUTE MATERIAL USING THE SAME

(71) Applicant: HUDENS BIO Co., Ltd, Gwangju (KR)

(72) Inventors: Riessa Nanda Mertamani, Gwangju (KR); Kim Hun, Daegu (KR); Kim Sukyoung, Gyeongsangbuk-do (KR)

(21) Appl. No.: 19/173,696

(22) Filed: Apr. 8, 2025

(30) Foreign Application Priority Data
Apr. 9, 2024 (KR) 10-2024-0048247

(51) Int. Cl. A61L 27/56; A61L 27/24; A61L 27/46

(52) U.S. Cl. CPC A61L 27/56; A61L 27/24; A61L 27/46; A61L 2430/02

(57) ABSTRACT

The present disclosure relates to a porous composite material, and more specifically to a porous composite material that is capable of manufacturing a porous composite material having a pore size that exhibits excellent bone regeneration ability, 5 while also having excellent compressive strength, clastic modulus and shape recovery properties, a manufacturing method thereof and a bone graft material using the same.

### CROSS-REFERENCE TO RELATED APPLICATION(S)

This application claims priority to and the benefit of Korean Patent Application No. 10-2024-0048247, filed on Apr. 9, 2024, the disclosure of which is incorporated herein by reference in its entirety.

### TECHNICAL FIELD

The present disclosure relates to a porous composite material, and more specifically to a porous composite material using a collagen porous composite material including a calcium phosphate-based ceramic material, a manufacturing method thereof and a bone graft material using the same.

### RELATED ART

Currently, various calcium phosphate ceramic materials are used as porous bone grafting materials, but there are still many problems to be solved in order to satisfy the clinical requirements in terms of mechanical strength, pore structure and bone regeneration ability. The pore size of artificially manufactured ceramic porous bodies for bone regeneration varies from 1 μm to about 10 mm, and the size and shape are diverse, and these characteristics vary depending on the manufacturing method.

The general method for manufacturing ceramic porous bodies is to mix a certain amount of a flux material with ceramic particles having a controlled size for the desired pore size, shape the same, melt the flux through high-temperature treatment, and agglomerate the ceramic particles to manufacture a ceramic porous body, or to obtain a porous body by partially sintering ceramic particles having a desired particle size at a temperature lower than the sintering temperature after compression molding. The pores are created from internal pores (micro pores) existing inside the particles and pores (macro pores) existing between the particles, and the size of the pores existing between the particles is related to the size of the raw material particles. These methods are not only difficult to effectively control the pore size and distribution of the porous body, but also have the problem of making it difficult to increase the porosity to 50% or more.

Therefore, in order to induce more efficient bone tissue regeneration than existing ceramic porous bodies, porous materials that exhibit biomimetic, biodegradable, three-dimensionally interconnected porous structures, appropriate mechanical properties and osteogenic induction and osteoconductivity are required. Traditional ceramic porous body manufacturing methods, such as coral or sponge replication, and sacrificial molding using polymers and salts as molds, are mainly used, and recently, porous bodies with three-dimensional porous structures, pore sizes, shapes and porosities using 3D printing have been made possible. However, due to the high-temperature sintering process, biocompatibility and osteoconductivity are reduced, and new bone regeneration is significantly lower than that of allogeneic or xenogeneic bones.

In order to solve these problems, porous ceramic-polymer composites are being attempted, and among these, collagen porous composite materials containing calcium phosphate ceramic materials are being studied extensively as they show the most excellent properties in terms of bone regeneration. Calcium phosphate powders added to the collagen porous matrix include hydroxyapatite (HA), β-tricalcium phosphate (β-TCP) and octacalcium phosphate (OCP).

HA is the main mineral component of teeth and bones in the body, and is used as a bone substitute because it fills the missing bone space and promotes new bone ingrowth, and it is mainly used as a coating material on the surface of artificial implants to improve osseointegration between the implant and the surrounding bone. However, artificial HA bone grafting materials have the problem of remaining as foreign substances in the body even after bone regeneration. Therefore, β-TCP or OCP is used as absorbable materials rather than bio-non-absorbable materials, and these materials utilize the characteristic of being completely absorbed after bone regeneration is completed in the bone defect area.

Recently, a patent has been filed for the manufacture of a porous body for bone regeneration containing an OCP material, which is a precursor of human HA mineral (biological apatite), in a collagen sponge-type matrix (see Korean Patent Laid-Open Publication No. 10-2017-0084323). In particular, the OCP-collagen porous bone regeneration material has excellent biocompatibility and it is a product that is absorbed after being inserted into the human body, thereby solving the problems of existing products. Recently, a manufacturing method that is capable of mass-producing expensive, high-purity OCP has been developed, thereby solving the economic feasibility problem that has been an obstacle to the commercialization of OCP-collagen porous bodies (see Patent 1: Korean Patent No. 10-2019-0044553).

The OCP-collagen porous composite material can solve the problems of existing ceramic porous bodies, such as desired porous body pore characteristics, porous body shape and shape restoration ability, and thus, it is expected that the OCP-collagen composite material can be conveniently used as a bone regeneration material for a wide range of bone defect areas. However, the related application patent (10-2017-0084323) had a problem in implementing a composite material including a pore size and sufficient OCP content to be able to exhibit bone regeneration ability required for clinical use. In addition, there is a problem in the compressive strength and shape recovery ability that are sufficient for application to granules at the desired pore size.

Accordingly, there is a need for a technology for manufacturing a bone graft material using an OCP-collagen porous composite material that includes an appropriate pore size and sufficient OCP content to be able to express the bone regeneration ability required in clinical practice, and has high compressive strength, a porous body shape and excellent shape recovery ability.

### RELATED ART DOCUMENTS

### Patent Documents

(Patent Document 1) Korean Patent Laid-Open Publication No. 10-2017-0084323
(Patent Document 2) Korean Patent Laid-Open Publication No. 10-2019-0044553

### SUMMARY

The bone graft material that is generally used is manufactured in the form of powder or granules to fill in the bone defect area, and the present disclosure relates to a method for manufacturing a product that is convenient to handle in clinical practice and promotes bone regeneration in the body by applying a bone defect area at once using a sponge-type organic/inorganic porous composite material. In particular, it is intended to provide a method for manufacturing a porous composite material that adds a desired pore size and OCP content to exhibit excellent bone regeneration ability, and has excellent compressive strength, elastic modulus, porous body shape and shape recovery ability sufficient for clinical application.

The problems to be solved by the present disclosure are not limited to the problems mentioned above, and other problems that are not mentioned will be clearly understood by those skilled in the art from the description below.

The present disclosure provides a method for manufacturing a porous composite material, including preparing a mixture by mixing octacalcium phosphate (OCP) particles and a collagen solution; preparing a dried body by freezing and freeze-drying the mixture; and manufacturing a porous composite material by performing high-temperature treatment on the dried body.

According to an embodiment of the present disclosure, the OCP particles may be surface-treated OCP particles that are classified into a size of 15 to 55 μm, and then subjected to surface treatment with a polymer.

In addition, the surface treatment may be performed by using a polymer including at least any one of carboxymethyl cellulose (CMC), collagen and hyaluronic acid.

In addition, the surface-treated OCP particles may be prepared by adding OCP particles to an aqueous solution having a CMC concentration of 0.1 to 2.0% to a concentration of 0.1 to 15%, stirring for 0.5 to 8 hours, freezing at a temperature of −100 to −70° C. for 3 to 9 hours, freeze-drying at a temperature of −80 to −20° C. for 60 to 100 hours, and then pulverizing.

In addition, the collagen solution may include collagen at a concentration of 0.6 to 2.0%, and have a pH of 6.8 to 7.6.

In addition, the mixture may be mixed with the collagen and OCP particles in a weight ratio of 5:95 to 15:85.

In addition, the preparing a dried body may be performed by freezing the mixture at −100 to −60° C. for 7 hours or more, and then freeze-drying at −40 to ˜10° C. for 20 to 28 hours, at −60 to −20° C. for 8 to 16 hours, and at −5 to 8° C. for 50 to 70 hours. In addition, the heat treatment of the porous dried body may be performed by heat treating the porous composite material at a temperature of 85 to 125° C. under reduced pressure conditions for 40 to 56 hours.

In addition, the present disclosure provides a porous composite material, including octacalcium phosphate (OCP) particles and collagen, wherein pores having a pore diameter of 15 to 110 μm account for 85% or more of the total pores.

According to an embodiment of the present disclosure, the OCP particles may have an average particle size of 15 to 55 μm, and the porous composite material may include the collagen and OCP particles in a weight ratio of 5:95 to 15:85.

In addition, the present disclosure provides a bone graft material using the above-described porous composite material.

The present disclosure provides a porous composite material and a method for manufacturing a bone graft material using the same. In particular, it is manufactured to have an appropriate pore size such that osteoblasts can easily enter the bone graft material and exhibit excellent bone regeneration ability, while simultaneously exhibiting excellent effects in compressive strength, elastic modulus and shape recovery such that it is easy to handle and apply clinically.

In addition, the porous composite material described above can be used as a bone graft material in the fields of dentistry, orthopedics and plastic surgery for bone defect repair, bone defect repair after craniotomy or open thoracotomy and the like. For example, in the field of dentistry, it can be applied to periodontal disease, cystic cavity, atrophic alveolar ridge, extraction socket and the like, and in the field of orthopedics, it can promote bone regeneration when it is applied to bone defects caused by trauma such as bone tumor resection and fracture. The porous composite material can be used to promote bone regeneration speed by containing cytokines (bone morphogenetic protein-2, transforming growth factor β1, etc.) that help bone formation.

### BRIEF DESCRIPTION OF THE DRAWINGS

FIG. 1 is a graph showing the results of measuring the pore size ratio of an OCP-collagen porous composite material according to an embodiment of the present disclosure.

FIG. 2 is a graph showing the results of measuring the compressive strength and elastic modulus of OCP-collagen porous composite materials (Example 1 and Comparative Example 1) according to an embodiment of the present disclosure.

FIG. 3 is a set of scanning electron microscope images of OCP-collagen porous composite materials (Example 1_a, b and Comparative Example 1_c, d) according to an embodiment of the present disclosure.

FIG. 4 is a set of images of osteoblast culture over time of an OCP-collagen porous composite material (Example 1) according to an embodiment of the present disclosure.

FIG. 5 is a process flow chart for manufacturing an OCP-collagen porous composite material according to an embodiment of the present disclosure.

### DETAILED DESCRIPTION OF THE EMBODIMENTS

Hereinafter, embodiments of the present disclosure will be described in detail so that those skilled in the art can easily practice the present disclosure. The present disclosure may be implemented in various different forms and is not limited to the embodiments described herein.

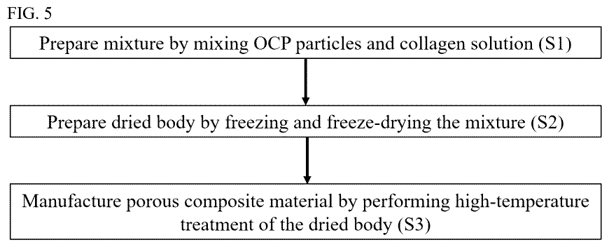

The porous composite material according to the present disclosure is manufactured by including step (S1) of preparing a mixture by mixing octacalcium phosphate (OCP) particles and a collagen solution, as shown in FIG. 5, step (S2) of freezing and freeze-drying the mixture to prepare a dried body, and step (S3) of manufacturing a porous composite material by performing high-temperature treatment on the dried body.

The step of preparing the OCP particles used in the present disclosure may use synthesized OCP particles as they are, or may use OCP particles that are surface-treated with a polymer to improve adhesion to a collagen matrix. The average size of the OCP particles may be 15 to 55 μm, and preferably, the average particle diameter may be in the range of 20 to 50 μm. Satisfying the above average size range may be more advantageous in achieving the objects of the present disclosure.

In this case, the polymer used for the polymer surface treatment may include at least any one of carboxymethyl cellulose (CMC), collagen and hyaluronic acid, and more preferably, CMC may be more advantageous in achieving the objects of the present disclosure. In an aqueous solution having a CMC concentration of 0.1 to 2.0%, OCP particles were stirred for 0.5 to 8 hours to a concentration of 0.1 to 15%, frozen at a temperature of −100 to −70° C. for 3 to 9 hours, and then freeze-dried at a temperature of −80 to −20° C. for 60 to 100 hours, and then pulverized by using a mortar to prepare CMC-coated OCP particles.

The collagen solution used for the manufacture of the OCP-collagen porous composite material may include collagen at a concentration of 0.6% or more, and preferably, at a concentration of 0.6 to 2%, and may have a pH of 6.8 to 7.6, and preferably, a pH of 7.0 to 7.4. If the collagen solution satisfies the collagen concentration and pH range, the mechanical stability of the porous composite material may be excellent, and the bone regeneration ability, compressive strength, elastic modulus and shape recovery may be excellent.

The collagen used at this time is a long fiber with three types of amino acids (glycine, proline, hydroxyproline) in a triple helix structure and is hardly dissolved in an aqueous solution, and thus, in the case of solid collagen, it may be dissolved in acetic acid and used. The origin and properties of the collagen are not particularly limited, and various collagens may be used. Preferably, it is obtained by solubilizing a protein-decomposing enzyme, such as pepsin or pronase, and enzyme-solubilized collagen from which telopeptides have been removed is used. As for the type of collagen, fibrous collagen types I, II, III and IV collagen are preferable, and type I collagen, which is included in large quantities in a living body, or a mixture of types I and III collagen is more preferable.

Although a specific animal-derived collagen is not specifically limited as a raw material, collagen derived from skin, bones, tendons and the like of pigs, cows and the like may be preferably used. Since collagen is a component derived from a living organism, it has the characteristic of high safety, and in particular, enzyme-solubilized collagen is preferable because it has low allergic reactivity. For example, the collagen may be a salt-precipitated compressed concentrated liquid collagen (LCol) derived from pig skin, and thus, it may be dissolved in an aqueous solution without using acetic acid and used as a collagen solution.

Step (S1) of preparing a mixture of the OCP particles and the collagen solution may mix the collagen and the OCP particles in a weight ratio of 5:95 to 15:85, and preferably, in a weight ratio of 8:92 to 13:87. The weight ratio represents the weight ratio between the content of collagen included in the collagen solution and the OCP particles. If the weight ratio of the collagen and OCP is less than 5:95 (collagen less than 5, OCP more than 95), the compressive strength, elastic modulus and shape recovery force may decrease, and if the weight ratio of the collagen and OCP exceeds 15:85 (collagen more than 15, OCP less than 85), the bone regeneration ability may decrease.

Step (S2) of preparing the dried body is performed by freezing and freeze-drying the mixture. Specifically, the process may be performed by freezing the mixture at a temperature of −100 to −60° C. for 7 hours or longer, and then freeze-drying at a temperature of −40 to ˜10° C. for 20 to 28 hours, at a temperature of −60 to −20° C. for 8 to 16 hours, or at a temperature of −5 to 8° C. for 50 to 70 hours, and preferably, freezing the mixture at a temperature of −90 to −70° C. for 8 hours or longer, and then freeze-drying at a temperature of −25 to ˜15° C. for 22 to 26 hours, at a temperature of −50 to −30° C. for 9 to 15 hours, or at a temperature of −3 to 6° C. for 55 to 65 hours. By satisfying the freezing condition range and the freeze-drying condition range for preparing the dried body, it may be more advantageous in achieving the objects of the present disclosure.

The step of performing heat treatment on the porous dried body may specifically perform heat treatment on the porous composite material at a temperature of 85 to 125° C. for 40 to 56 hours under reduced pressure conditions, and preferably at a temperature of 95 to 115° C. for 43 to 53 hours. When the ranges of conditions of reduced pressure, temperature and time during the heat treatment are satisfied, it may be more advantageous to achieve the objects of the present disclosure. Meanwhile, preferably, the reduced pressure condition is performed by setting the negative pressure to −1.5 MPa to −0.5 MPa using a vacuum pump.

The porous composite material manufactured by the present disclosure includes OCP particles and collagen, and while the OCP particles are dispersed and scattered in the collagen porous matrix, as a result of measuring the pore characteristics by mercury intrusion porosimetry, pores having a pore diameter of 15 to 110 μm account for 85% or more of the total pores, and preferably, pores having a pore diameter of 20 to 100 μm may account for 85% or more of the total pores. When the pore diameter accounting for 85% or more of the total pores of the porous composite material is less than 15 μm, the compressive strength increases but the bone regeneration ability decreases, and when it exceeds 110 μm, the bone regeneration ability improves but the compressive strength or shape recovery ability may decrease. The porous composite material may have a porosity of 55% or more, and preferably, a porosity of 60% or more. If the porosity is less than 55%, the bone regeneration ability decreases but the compressive strength increases. If it is over 60%, the result will show the opposite trend to the case of less than 55%.

The compressive strength and elastic modulus of the porous composite material were measured by manufacturing a cylindrical sample according to the ASTM F1538-03 (2017) method, and the measured compressive strength and elastic modulus may be 0.10 MPa or more and 2.6 MPa or more, respectively, and preferably, the compressive strength and elastic modulus may be 0.16 MPa or more and 3.5 MPa or more, respectively. Additionally, in order to measure the shape recovery force of the porous composite material, it was processed into a cylindrical shape, compressed to a diameter of 50%, and then immersed in a phosphate buffer solution to measure the time it takes for it to recover to its initial diameter. The shape recovery time measured by the above measurement method may be 4 seconds or less, and preferably, 3 seconds or less. That is, since the manufactured porous composite material satisfies all of the ranges of compressive strength, elastic modulus and shape recovery force required as a bone regeneration material, it is expected to simultaneously exhibit excellent bone regeneration ability as well as excellent compressive strength, elastic modulus and shape recovery force.

Hereinafter, the present disclosure will be described in more detail through examples. However, the present disclosure should not be interpreted as limited by the following examples.

### EXAMPLE

### Example 1

OCP particles having an average particle size of 35 μm were placed in a container of a CMC solution having a concentration of 0.5% to a concentration of 3.3%, stirred for 1 hour, frozen at a temperature of −80° C. for 6 hours, freeze-dried at a temperature of −55° C. for 72 hours, and then pulverized by using a mortar to prepare CMC-coated OCP particles (surface-treated OCP). Then, the salt-precipitated compressed concentrated liquid collagen (type I collagen, Eubiosis) derived from pig skin was diluted to a concentration of 3% by using a phosphate buffer solution to prepare a collagen solution having a pH of 7.2.

A mixture of the collagen and the CMC-coated OCP was prepared at a weight ratio of 10:90, and then poured into a mold to create a desired shape. The mixture to which this shape was imparted was frozen at a temperature of −80° C. for 12 hours, and then freeze-dried at a temperature of −20° C. for 24 hours, at a temperature of −40° C. for 12 hours, and at a temperature of 1.5° C. for 60 hours to prepare a dried body. The dried body was separated from the mold, and heat-treated at a temperature of 105° C. for 48 hours under a negative pressure of −1.0 MPa using a vacuum pump to manufacture a desired porous composite material.

### Comparative Example 1

The salt-precipitated compressed concentrated liquid collagen derived from pig skin (type I and type III collagen, manufactured by Nippon Ham Co., Ltd., NMP collagen PS) was added to a phosphate buffer solution at a concentration of 3%, and a sodium hydroxide aqueous solution was added to prepare a collagen solution having a pH of 7.4. After preparing a mixture with OCP powder having an average particle size of 400 μm in the collagen solution at a weight ratio of 10:90, the mixture was placed in a centrifugal bottle, and centrifugation was performed for 20 minutes by using a centrifuge (TOMI Seiko Co., Ltd., GRX-250). Afterwards, the upper layer was discarded such that the collagen in the mixture was 3 wt %, and the lower contents were mixed with a spoon for about 2 minutes to obtain a composite gel.

This gel was placed in a cylindrical plastic container (inner diameter 8.5 mm, volume about 3.0 cm3) and degassed by using a centrifuge for 1 minute. After sealing the container, it was rapidly freeze-dried by immersing in methanol cooled to −80° C. After opening the stopper of the container, the frozen body was dried in a freeze dryer (−10° C., 48 hours). Next, after heat dehydration crosslinking was performed in a vacuum dryer (negative pressure-1.0 MPa, 150° C., 24 hours), it was cut to a thickness of 15 mm. The dried body made into the desired shape was sterilized by electron beam irradiation (15 kGy).

### Experimental Example 1

The following items were evaluated for Example 1 and Comparative Example 1 above.

### (1) Measurement of Mercury Intrusion Volume by Pore Size

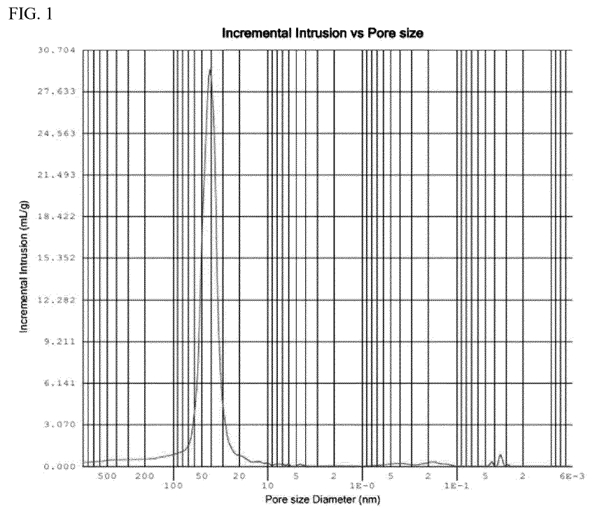

A sample of 0.7*0.7*0.7 mm in size was made for the porous composite material according to Example 1 above, and the mercury intrusion ratio by pore size was measured through the mercury intrusion porosimetry device (PM33GT, Quantachrome). In this case, the measurement condition was Hg (mercury) with a surface tension of 480.00 erg/cm², and the measurement was performed at 38° C. for 2 hours and 30 minutes under the condition of 1.5 bar (44.3 inHg). The results are shown in FIG. 1. As can be seen in FIG. 1, it can be confirmed that the porous composite material according to Example 1 had pores with a pore diameter of 15 to 110 μm accounting for 85% or more of the total pores.

### (2) Scanning Electron Microscope Analysis

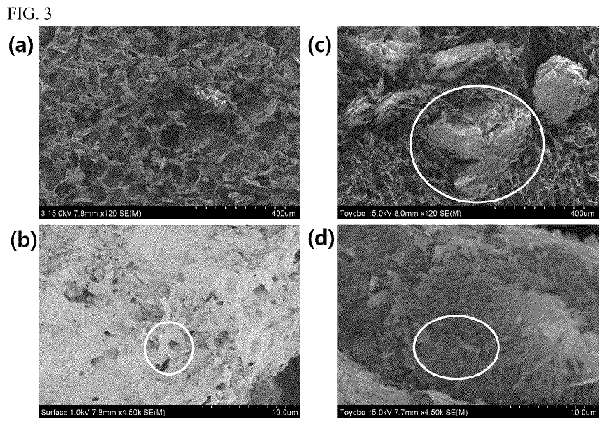

For the porous composite materials according to Example 1 and Comparative Example 1 above, SEM analysis was performed at 120 times magnification under 15.0 KV conditions and 4,500 times under 1.0 KV conditions using a scanning electron microscope (SEM) (S-4800, HITACH), respectively. The results are shown in FIG. 3. FIGS. 3a and 3b are SEM images of the porous composite material according to Example 1, and FIGS. 3c and 3d are SEM images of the porous composite material according to Comparative Example 1.

As can be seen in FIG. 3, the porous composite material according to Example 1 shows that small-sized (5-35 μm) OCP particles (grains) were uniformly distributed in the collagen scaffold at relatively low magnification (FIG. 3a), and it was confirmed that the small OCP particles (approximately 5 μm) had a plate-like structure, which is a unique shape, in the SEM image at relatively high magnification (FIG. 3b). However, the porous composite material according to Comparative Example 1 shows that relatively large-sized OCP lump (40-400 μm) particles were distributed in the collagen scaffold at relatively low magnification (FIG. 3c), and it was confirmed that the small OCP particles (approximately 5 μm) within the large particles had a plate-like structure, which is a unique shape, in the SEM image at relatively high magnification (FIG. 3d).

### (3) Evaluation of Cell Compatibility of Composite Materials

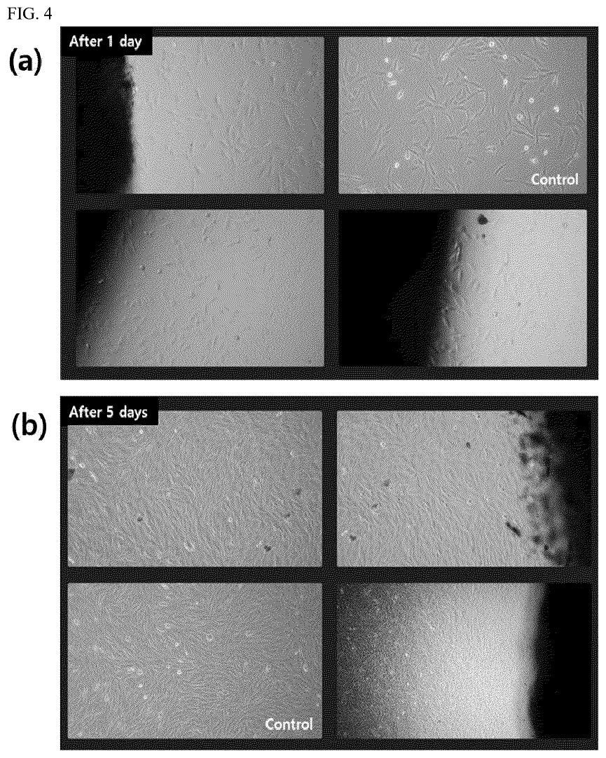

For the porous composite material according to Example 1 above, osteoblasts were cultured at 5*10³ cells/mL as shown in FIG. 4, and the compatibility between the composite material and osteoblasts was determined by using a microscope on day 1 (FIG. 4. (a)) and day 5 (FIG. 4. (b)).

As can be seen in FIG. 4, osteoblasts can be seen to increase significantly over time. This result shows the excellent cell compatibility of the porous composite material according to the present disclosure, and it can be seen that it satisfied the first essential condition required for bone regeneration. In addition, since osteoblasts grew vigorously in the area in contact with the porous composite material, it can be seen that osteoblasts can grow not only on the surface of the implant but also inside the porous characteristics.

### Examples 2 to 7

The same procedure as in Example 1 was followed, except that the collagen and OCP weight ratio, OCP average particle size, collagen concentration in the solution and polymer surface treatment of the OCP particles were changed to manufacture porous composite materials as shown in Tables 1 and 2 below.

### Experimental Example 2

The results of the evaluation of the characteristics of the porous composite materials according to Examples 1 to 7 and Comparative Example 1 are shown in Tables 1 and 2. The pore size and pore fraction, SEM images and cytocompatibility evaluation methods were mentioned above, and the shape recovery force measurement method was as follows.

### (1) Measurement of Compressive Strength and Elastic Modulus

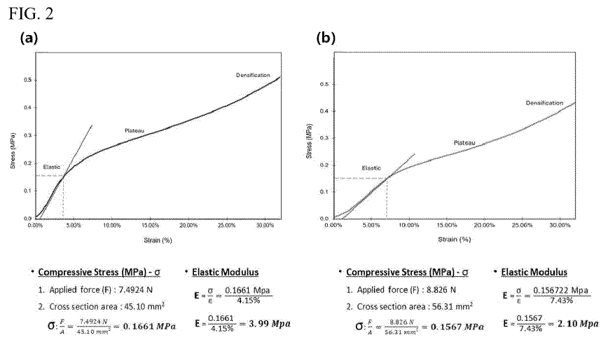

The compressive strength and elastic modulus of the porous composite materials according to the above examples and comparative examples were measured according to the ASTM F1538-03 (2017) method. In this case, the results for Example 1 and Comparative Example 1 are shown in FIG. 2. As can be seen in FIG. 2, the porous composite material of Comparative Example 1, which did not satisfy the constitutions of the present disclosure, had a compressive strength of 0.1567 MPa and an elastic modulus of 2.10 MPa (FIG. 2b), whereas the porous composite material according to Example 1 had a compressive strength of 0.1661 MPa and an elastic modulus of 3.99 MPa, which showed significantly superior compressive strength and elastic modulus (FIG. 2a).

### (2) Measurement of Shape Recovery Force

For the porous composite materials according to the above examples and comparative examples, the measured shape recovery force was measured by processing each porous composite material into a cylindrical shape with a diameter of about 8 mm, compressing and deforming the same to a diameter of 50% or less, and then immersing the same in a room temperature phosphate buffer solution to restore the same to its original size. The shape recovery rate was measured as the ratio of the size (diameter) at 30 seconds compared to the initial size.

TABLE 1

| Classification | | Example 1 | Example 2 | Example 3 | Example 4 |
|---|---|---|---|---|---|
| Weight Ratio of Collagen | | 10:90 | 3:97 | 25:75 | 10:90 |
| and OCP | | | | | |
| Size of OCP Particles | | 35 | 35 | 35 | 10 |
| (μm) | | | | | |
| Concentration of | | 3 | 3 | 3 | 3 |
| Collagen in | | | | | |
| Solution (%) | | | | | |
| OCP Surface Treatment | | Treated | Treated | Treated | Treated |
| Compressive Strength | | 0.166 | 0.170 | 0.154 | 0.142 |
| (MPa) | | | | | |
| Elastic Modulus (MPa) | | 3.99 | 3.72 | 3.95 | 3.90 |
| Shape | Time | 2 | 31 | 5 | 17 |
| Recovery | (Sec) | | | | |
| | Recovery | 103 | 97 | 101 | 101 |
| | Rate (%) | | | | |

TABLE 2

| | | | | | Compar- |
|---|---|---|---|---|---|
| | | | | | ative |
| Classification | | Example 5 | Example 6 | Example 6 | Example 1 |
| Weight Ratio of Collagen | | 10:90 | 10:90 | 10:90 | 10:90 |
| and OCP | | | | | |
| Size of OCP Particles | | 65 | 35 | 35 | 400 |
| (μm) | | | | | |
| Concentration of | | 3 | 0.5 | 3 | — |
| Collagen in | | | | | |
| Solution (%) | | | | | |
| OCP Surface Treatment | | Treated | Treated | Not | Not |
| | | | | treated | treated |
| Compressive Strength | | 0.139 | 0.112 | 0.098 | 0.156 |
| (MPa) | | | | | |
| Elastic Modulus (MPa) | | 2.57 | 3.06 | 3.09 | 2.10 |
| Shape | Time | 21 | 18 | 9 | >30 |
| Recovery | (Sec) | | | | |
| | Recovery | 103 | 102 | 102 | 89 |
| | Rate (%) | | | | |

As can be seen from Tables 1 and 2 above, it can be confirmed that the results of Example 1, which satisfied all of the weight ratio of collagen and OCP, average particle size of OCP, collagen concentration in the solution and polymer surface treatment according to the present disclosure, simultaneously exhibited the effects of significantly superior compressive strength and elastic modulus, significantly shorter shape recovery time, and significantly superior shape recovery rate, compared to Examples 2 to 7 and Comparative Example 1, which did not satisfy even one of these.

Although one embodiment of the present disclosure has been described above, the scope of the patent claims of the present disclosure is not limited to the embodiments presented in this specification, and should be interpreted to encompass various modified examples that can be obviously derived from the same by those skilled in the art. The scope of the patent claims is intended to encompass such modified examples.

What is claimed is:

1. A method for manufacturing a porous composite material, comprising:
preparing a mixture by mixing octacalcium phosphate (OCP) particles and a collagen solution;
preparing a dried body by freezing and freeze-drying the mixture; and
manufacturing a porous composite material by performing high-temperature treatment on the dried body.

2. The method of claim 1, wherein the OCP particles are surface-treated OCP particles that are classified into a size of 15 to 55 μm, and then subjected to surface treatment with a polymer.

3. The method of claim 2, wherein the surface treatment is performed by using a polymer comprising at least any one of carboxymethyl cellulose (CMC), collagen and hyaluronic acid.

4. The method of claim 3, wherein the surface-treated OCP particles are prepared by adding OCP particles to an aqueous solution having a CMC concentration of 0.1 to 2.0% to a concentration of 0.1 to 15%, stirring for 0.5 to 8 hours, freezing at a temperature of −100 to −70° C. for 3 to 9 hours, freeze-drying at a temperature of −80 to −20° C. for 60 to 100 hours, and then pulverizing.

5. The method of claim 1, wherein the collagen solution comprises collagen at a concentration of 0.6 to 2.0%, and has a pH of 6.8 to 7.6.

6. The method of claim 1, wherein the mixture is mixed with the collagen and OCP particles in a weight ratio of 5:95 to 15:85.

7. The method of claim 1, wherein the preparing a dried body is performed by freezing the mixture at −100 to −60° C. for 7 hours or more, and then freeze-drying at −40 to −10° C. for 20 to 28 hours, at −60 to −20° C. for 8 to 16 hours, and at −5 to 8° C. for 50 to 70 hours.

8. The method of claim 1, wherein the heat treatment of the porous dried body is performed by heat treating the porous composite material at a temperature of 85 to 125° C. under reduced pressure conditions for 40 to 56 hours.

9. A porous composite material, comprising octacalcium phosphate (OCP) particles and collagen,
wherein pores having a pore diameter of 15 to 110 μm account for 85% or more of the total pores.

10. The porous composite material of claim 9, wherein the OCP particles have an average particle size of 15 to 55 μm, and
wherein the porous composite material comprises the collagen and OCP particles in a weight ratio of 5:95 to 15:85.

11. A bone graft material using the porous composite material according to claim 9 or 10.
